(19) United States
(12) Patent Application Publication
(10) Pub. No.: US 2009/0177098 A1
(43) Pub. Date: Jul. 9, 2009

(54) Examination System and Examination Method

(76) Inventors: Kousuke Yakubo, Hokkaido (JP); Kazuhiko Yoshida, Hokkaido (JP); Hitoshi Fujii, Fukuoka (JP); Kazuhiko Oka, Hokkaido (JP); Satoshi Tanda, Hokkaido (JP); Ryuuji Morita, Hokkaido (JP)

Correspondence Address: MOORE & VAN ALLEN PLLC, P.O. BOX 13706, Research Triangle Park, NC, 27709, US

(21) Appl. No.: 12/087,930

(22) PCT Filed: Dec. 8, 2006
(86) PCT No.: PCT/JP2006/325117

§ 371 Date: Nov. 6, 2008

(30) Foreign Application Priority Data
Jan. 16, 2006 (JP) 2006-006976

(51) Int. Cl. A61B 5/02

(52) U.S. Cl. 600/504

(57) ABSTRACT

The blood flow is examined by making multifractal analysis of a blood flow velocity distribution in a vascular network and detecting a deviation of the blood flow velocity distribution from the multifractal distribution. The blood flow velocity distribution is provided as an image by irradiating laser light to the vascular network, converging, by an imaging lens, scattered laser light rays by blood cells in the blood flowing through blood vessels, detecting, by a photodetector, a speckle pattern produced owing to random interference between the scattered laser light rays and calculating the rate of change with time lapse of each speckle in the speckle pattern.

### TECHNICAL FIELD

The present invention generally relates to an examination system and examination method for examining the blood flow in a vascular network, and more particularly, to an examination system and examination method suitable for use in the diagnosis of any disease with abnormal blood flow in his or her vascular network.

### BACKGROUND ART

In the past, many eye diseases and diseases with abnormality in the ocular fundus have been diagnosed empirically by the doctors through the physiological function tests (refraction, adjustment, color sensation, light perception, eye position, ocular movement, intraocular pressure), slit-lamp microscopy, funduscopy, perimetry, fluorescein fundus angiography, electrophysiological study, etc.

With the above conventional methods of examination, however, the diagnosis takes much time and also the result of diagnosis varies from one doctor to another in not a few cases.

On the other hand, there has been developed “Laser Speckle Flowgraphy” to measure and image the blood flow in a living body in noncontact, noninvasive manner. Ocular fundus flowgraphy systems having the laser speckle flowgraphy applied therein are already commercially available (will be known by access to an Internet site “URL: http://leo10.cse.kyutech.ac.jp/lsfg.html” (as searched on Jan. 5, 2006), for example). As shown in FIG. 1, according to the laser speckle flowgraphy, laser light 101 is irradiated to the surface of a living body. The laser light 101 is scattered by scatterers (blood cells) 102 in the blood flowing through blood vessels. The scattered light rays 103 from the scatterers 102 are converged by an imaging lens 104. The scattered light rays 103 thus converged by the imaging lens 104 will randomly interfere with each other to produce a speckle pattern 105. This speckle pattern 105 is detected by an image sensor 106. By calculating the rate of change with time lapse of each speckle in the speckle pattern 105, it is possible to provide a distribution of blood flow velocity as an image (two-dimensional map). Therefore, it is considered that the laser speckle flowgraphy is used to diagnose eye diseases and diseases with abnormality in the ocular fundus.

However, since the diagnosis, made based on such images produced through the laser speckle flowgraphy, of eye diseases and diseases with abnormality in the ocular fundus depends greatly upon the doctor's experiences, the result of diagnosis varies from one doctor to another in many cases.

Therefore, a subject to be solved by the invention is to provide an examination system and examination method permitting the doctor to examine the blood flow in the vascular network simply and accurately in a noncontact, noninvasive manner and make a diagnosis accurately and easily with any other method of examination employed in combination depending upon the presence or absence, and extent in seriousness, of an abnormal blood flow found through the noncontact, noninvasive examination.

### DISCLOSURE OF THE INVENTION

The Inventors of the present invention were dedicated to solving the above-mentioned subject by topological approach. With attention focused on the effectiveness of the multifractal analysis, the Inventors actually made multifractal analysis of the distribution of blood flow velocity in the choroid vascular network of eye. The result of multifractal analysis proved that when the blood flow in the choroid vascular network was normal, the distribution of blood flow velocity could be regarded as a substantial multifractal distribution and that when the blood flow was abnormal, the blood flow velocity distribution deviated from the multifractal distribution. The Inventors made further studies. The results of the further studies revealed that the above findings were also true with many other vascular networks including the capillary network, and thus the Inventors worked out the present invention.

The multifractal will be explained simply below (also see “Fractal Concepts in Condensed Matter Physics” by T. Nakayama and K. Yakubo, Springer-Verlag, 2002, p. 180). The fractal has a self-similar structure having no characteristic length. The self-similar structure can be quantified with a fractal dimension (D_{f}). The “Sierpinski Gasket” is illustrated as a well-known example of the fractal in FIG. 2. On the assumption that as in FIG. 2,

M=aL^{D}^{_{f}} (1)

the following will result:

$${a ( \frac{L}{2} )}^{{D}_{f}} = \frac{1}{3} M = \frac{1}{3} {aL}^{{D}_{f}} \qquad ( 2 )$$

Therefore, the fractal dimension D_{f} will be given as follows:

$${D}_{f} = \frac{log 3}{log 2} \approx 1.58 \qquad ( 3 )$$

The multifractal has a distribution (μᵢ) having no characteristic length and variable in fractal dimension from one strength to another of the distribution. The multifractal distribution can be quantified with a multifractal spectrum f(α) which is an infinite fractal dimension set. Here is assumed a square area of which one side has a length L as shown in FIG. 3, and the square area is divided into sections, that is, boxes, of which one side has a length l. The box measure will be given as follows:

$${μ}_{b ( l )} = {\sum }_{i ∈ {b}_{j} ( l )} {μ}_{i} \qquad ( 4 )$$

The q-th order moment of the box measure will be given as follows:

$${Z}_{q} ( l ) ≡ {\sum }_{b} {( {μ}_{b ( l )} )}^{q} \qquad ( 5 )$$

In case the distribution is a multifractal one, the following will result:

Z_{q}(l)∝l^{τ(q)} (6)

where τ(q) is a mass exponent.

For the multifractal, a generalized dimension is defined as follows:

$${D}_{q} = \frac{τ ( q )}{q - 1} \qquad ( 7 )$$

The multifractal spectrum is represented as follows by the Legendre transformation with the equations given below:

$$\begin{matrix}f ( α ) = α q - τ ( q ) & ( 8 ) \\ α = d τ ( q ) / d q & ( 9 )\end{matrix}$$

However, the calculation by the Legendre transformation is poor in accuracy since it includes a numerical differentiation. On this account, a q-microscope as given below:

$${m}_{b ( l )} ( q ) = \frac{{μ}_{b ( l )}^{q}}{{\sum }_{{b}^{′}} {μ}_{{b}^{′ ( l )}}^{q}} \qquad ( 10 )$$

should preferably be used for an improved accuracy of the actual calculation and the multifractal spectrum be calculated with the following:

$$\begin{matrix}f ( α ) = \frac{{\sum }_{b} {m}_{b ( l )} ( q ) log {m}_{b ( l )} ( q )}{log l} & ( 11 ) \\ α = \frac{{\sum }_{b} {m}_{b ( l )} ( q ) log {μ}_{b ( l )}}{log l} & ( 12 )\end{matrix}$$

One typical example of the distributions known as a multifractal distribution is the distribution of critical wave function in the metal-insulator transition. One example of the critical wave function distributions is shown in FIG. 4A, and a multifractal spectrum of this distribution is shown in FIG. 4B. As will be seen in FIG. 4B, the multifractal spectrum is characterized by its pseudo-parabolic shape symmetric with respect to a straight line of α≈2.2. For comparison with this multifractal spectrum, a random distribution is shown in FIG. 5A as one example of non-multi-fractal distributions, and a multifractal spectrum of the random distribution is shown in FIG. 5B. As will be seen in FIG. 5B, the multifractal spectrum has an asymmetric, non-parabolic shape.

To solve the above-mentioned subject, according to a first invention, there is provided an examination system for examining the blood flow in a vascular network, wherein the blood flow is examined by multifractal analysis of the blood flow velocity distribution in the vascular network.

Typically, multifractal analysis is made of the blood flow velocity distribution in the vascular network of a test object and a deviation of the blood flow velocity distribution from the multifractal distribution is detected, to thereby examine the blood flow and determine the presence or absence, and extent in seriousness, of an abnormal blood flow. For getting a distribution of blood flow velocity in the vascular network, the laser speckle flowgraphy should preferably be used. In addition, there may be used the DGV (Doppler Global Velocimeter) method in which the Doppler effect and a special optical filter (absorption line filter) are used in combination to visualize a two-dimensional velocity field as image contrast, PIV (Particle Image Velocimeter) method in which particles in a plane are exposed to light for a short time to track their movement, laser induced fluorescence method in which laser light is irradiated to a fluorescence dye for excitation and light emission and the velocity field is captured as fluorescence intensity or the like. The laser Doppler velocimeter method may be used as the case may.

The vascular network of the test object may basically be various vascular networks including capillary networks in all bodily regions. The test object may basically be any animals including human beings and animals other than the human beings. The test object is typically an animal having a closed blood-vascular system (closed circulatory system). Such an animal is for example a vertebrate. It is a mammal among others. The vascular networks of the human being include, for example, the choroid vascular network of eye, retinal vascular network, vascular network in the upper bodily portion, pulmonary vascular network, hepatic vascular network, gastric vascular network, splenic vascular network, intestinal vascular network, kidney vascular network, vascular network in the lower bodily portion, etc.

Also, according to a second invention, there is provided an examination system for examining the blood flow in a vascular network, the system comprising:

a laser source to irradiate laser light to the vascular network;

a photodetector to detect scattered light rays resulted from irradiation of the laser light to the vascular network; and

an arithmetic unit for determining a blood flow velocity distribution in the vascular network on the basis of an output signal from the photodetector and making multifractal analysis of the blood flow velocity distribution to detect a deviation of the blood flow velocity distribution from a multifractal distribution.

A laser source may appropriately be selected correspondingly to an animal under examination, region of interest, etc. The laser source may be of any type. Generally, a laser source which can generate laser light having a wavelength band ranging from near-infrared light to visible light is used. Also, the photodetector may be of any type and any appropriate one may be selected as necessary. Specifically, the photodetector is a two-dimensional image sensor (CCD sensor, MOS sensor, image pickup tube or the like). The arithmetic unit may be a computer. Results of computation from the arithmetic unit are displayed numerically or graphically on a display or printed out by a printer, whichever may be selected as necessary.

The aforementioned description of the first invention is also true for other than described above of the second invention.

Also, according to a third invention, there is provided an examination method for examining the blood flow in a vascular network, wherein the blood flow is examined by multifractal analysis of the blood flow velocity distribution in the vascular network.

The aforementioned description of the first invention is also true for other than described above of the third invention.

### BRIEF DESCRIPTION OF THE DRAWINGS

FIG. 1 is a schematic diagram for explaining the laser speckle flowgraphy.

FIG. 2 is a schematic diagram for explaining the fractal.

FIG. 3 is a schematic diagram for explaining the multifractal.

FIGS. 4A and 4B are schematic diagrams showing an example of the distribution of critical wave function in the metal-insulator transition and a multifractal spectrum of the distribution.

FIGS. 5A and 5B are schematic diagrams showing an example of the random distribution and a multifractal spectrum of the random distribution.

FIG. 6 is a schematic diagram showing an examination system according to one embodiment of the present invention.

FIG. 7 is a schematic diagram for explaining the meanings of three quantities αₘᵢₙ, αₘₐₓ and α₀ as a base for evaluation of the multifractal property.

FIG. 8 is a horizontal sectional view of the eyeball.

FIG. 9 is a fragmentary sectional view showing the retina, choroid and sclera.

FIG. 10 is a schematic diagram showing an example of the choroid vascular network.

FIG. 11 is a photograph as a substitution for drawing showing an example of a fundus camera-captured ocular fundus image.

FIG. 12 is a schematic diagram for explaining the evaluation order q_{w} and evaluation function width w.

FIGS. 13A, 13B, 13C and 13D are photographs as substitutions for drawing showing ocular fundus images of examinees A to D with normal eyes, each with values of evaluation indexes 1 to 3.

FIGS. 14A, 14B, 14C and 14D are photographs as substitutions for drawing showing ocular fundus images of examinees E to H with normal eyes, each with values of evaluation indexes 1 to 3.

FIGS. 15A, 15B, 15C and 15D are photographs as substitutions for drawing showing ocular fundus images of examinees 1 to 4 with AMD disease at both eyes, each with value of evaluation indexes 1 to 3.

FIGS. 16A and 16B are photographs as substitutions for drawing showing an ocular fundus image of an examinee 5 with AMD disease at one eye, the image being of the other eye with no AMD, and an ocular fundus image of an examinee with PIC disease, with evaluation indexes 1 to 3.

FIG. 17 is a graph showing evaluation indexes 1 to 3 of the examinees A to H with normal eyes, examinees 1 to 5 with AMD disease, and examinee with PIC disease.

FIG. 18 is a schematic diagram showing the multifractal spectrum of the examinee E with normal eyes.

FIG. 19 is a schematic diagram showing the multifractal spectrum of the examinee 1 with AMD disease.

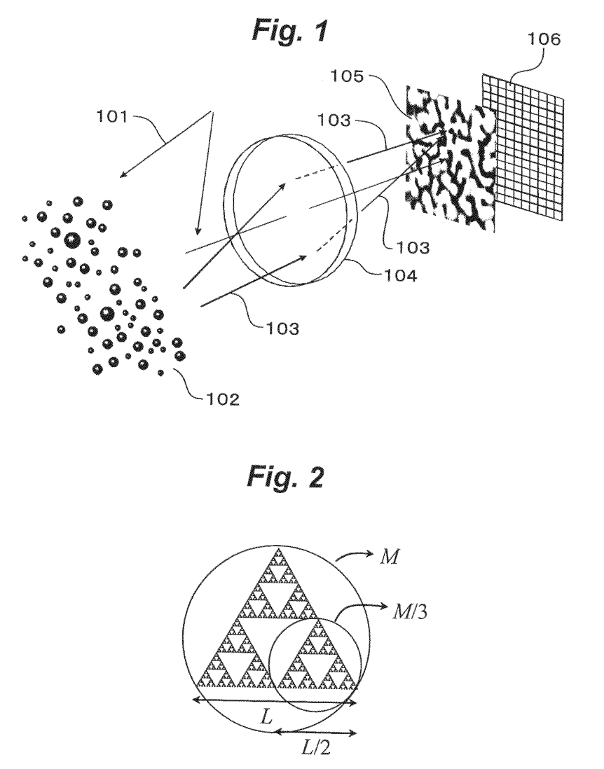

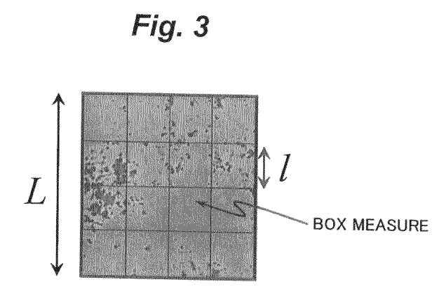

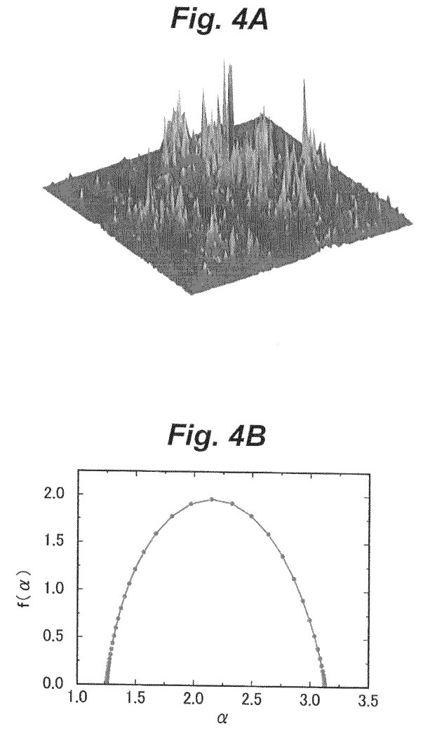

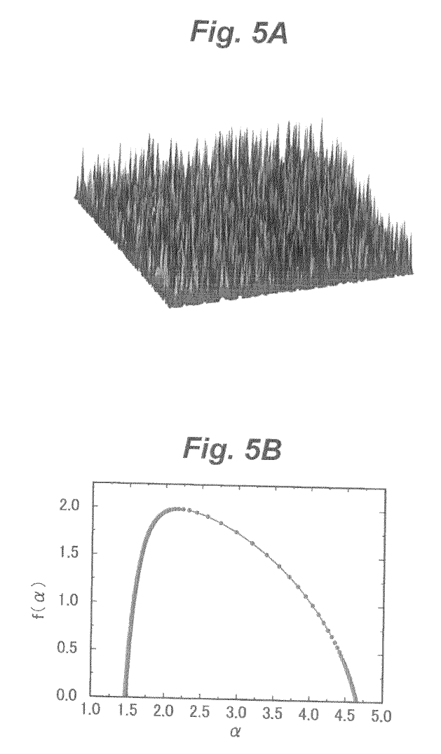

### BEST MODE FOR CARRYING OUT THE INVENTION

The present invention will be described in detail below concerning one embodiment thereof with reference to the accompanying drawings.

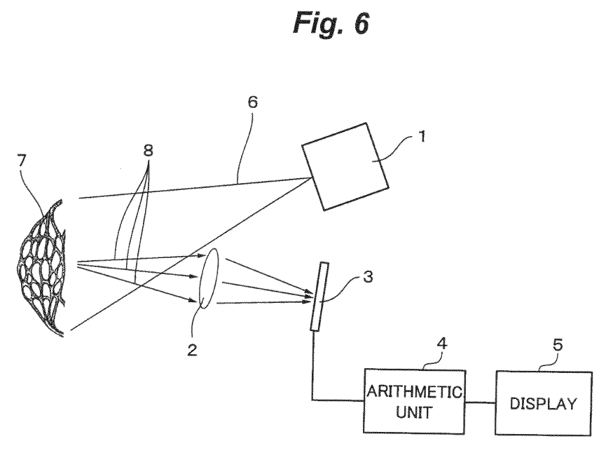

FIG. 6 shows an examination system according to the embodiment of the present invention. In this examination system, the laser speckle flowgraphy is used to measure the distribution of blood flow velocity in the vascular network. As shown in FIG. 6, the examination system includes a laser light source 1, imaging lens 2, photodetector 3, arithmetic unit 4 and display 5.

In the above examination system, laser light 6 emitted from the laser light source 1 is irradiated to a vascular network 7 in a region of interest of a test object and scattered by blood cells in the blood flowing through the vascular network 7. Scattered light rays 8 are converged by the imaging lens 2 to produce speckles (not shown). The speckles are detected by the photodetector 3. An analog signal from the photodetector 3 is converted into a digital signal by analog-to-digital conversion. The digital signal is calculated in the arithmetic unit 4 to obtain the distribution of blood flow velocity in the vascular network 7. Multifractal analysis is made using data the blood flow velocity distribution thus obtained has. The display 5 can display the blood flow velocity distribution as an image (two-dimensional map) and readable numeric data, and also the result of multifractal analysis as a multifractal spectrum and a digitized deviation of the multifractal spectrum from a multifractal distribution.

### Example

As the examination system including the laser light source 1, imaging lens 2, photodetector 3, arithmetic unit 4 and display 5, there was adopted the commercially available ocular fundus flowgraphy system using the laser speckle flowgraphy (will be known by access to an Internet site “URL: http://leo10.cse.kyutech.ac.jp/lsfg.html” (as searched on Jan. 5, 2006), for example). In this ocular fundus flowgraphy system, the fundus camera includes the laser light source 1, imaging lens 2 and photodetector 3. As the laser light source 1, there was used a semiconductor laser of which the emission wavelength is 830 nm and which can generate laser light 6 whose wavelength is in the near-infrared region. As the photodetector 3, there was used a two-dimensional CCD image sensor. As the computation unit 4 and display 5, there was used a commercially available personal computer system. The hard disk in the personal computer body had stored therein a laser speckle flowgraphy program, a program that outputs a blood flow velocity distribution as a numerical value proportional to a velocity value in a format such as CSV (Comma Separated Value) and a multifractal analysis program. The multifractal spectrum was calculated by a method using the aforementioned equations (10) to (12) for an improved accuracy of calculation.

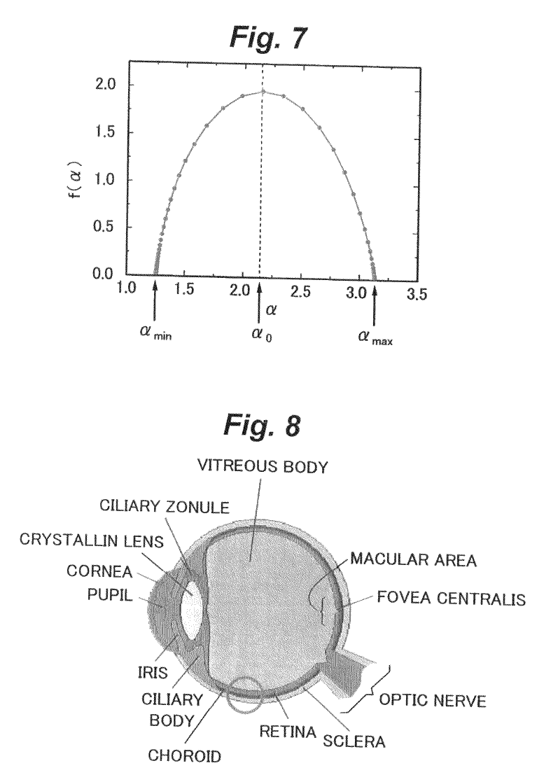

For quantitative evaluation of the multifractal property of a blood flow velocity distribution, three evaluation indexes are used. FIG. 7 explains the meanings of three quantities αₘᵢₙ, αₘₐₓ and α₀ as a base for evaluation of the multifractal property.

The evaluation index 1 indicates how much α₀ deviates from the midpoint of [αₘᵢₙ, αₘₐₓ] and it is defined as follows:

$$Evaluation index 1 = \vert \frac{2 {α}_{0} - {α}_{max} - {α}_{min}}{{α}_{max} - {α}_{min}} \vert \qquad ( 13 )$$

When α₀ is completely coincident with the midpoint of [αₘᵢₙ, αₘₐₓ] (that is, in case the multifractal property is good), the evaluation index 1=0. When α₀ is completely deviant from the midpoint of [αₘᵢₙ, αₘₐₓ] (that is, in case the multifractal property is very poor and α₀=αₘₐₓ or α₀=αₘᵢₙ), the evaluation index 1=1.

The evaluation index 2 indicates how great the deviation between the following equations (14) and (15) is:

$$\begin{matrix}{S}_{low} = {\int }_{{α}_{min}}^{{α}_{0}} f ( α ) d α & ( 14 ) \\ {S}_{high} = {\int }_{{α}_{0}}^{{α}_{max}} f ( α ) d α & ( 15 )\end{matrix}$$

and it is used to evaluate the extent of symmetry of f(α). The evaluation index 2 is defined as follows:

Evaluation index 2=

$$\vert \frac{{S}_{high} - {S}_{low}}{{S}_{high} + {S}_{low}} \vert \qquad ( 16 )$$

Also in this case, when f(α) has a complete symmetry, the evaluation index 2=0. When f(α) has a complete asymmetry (that is, either S_{high} or S_{slow} is zero), the evaluation index 2=1.

As above, the evaluation indexes 1 and 2 depend upon the symmetry of the multifractal spectrum f(α), while the evaluation index 3 is a quantified deviation of f(α) from a theoretical formula. It should be noted that the “theoretical formula” means a generalized theoretical formula for a potential difference distribution in a hierarchical resistance network in which f(α) is theoretically determined.

The multifractal spectrum f(α) for the potential difference distribution in a hierarchical resistance network is given by the following equation (17) (as in “Fractal Concepts in Condensed Matter Physics” by T. Takayama and K. Yakubo, Springer-Verlag, 2002, p. 180):

$$f ( α ) = \frac{1}{ν} - \frac{1}{v log 2} [ ( \frac{log 6}{log 2} - α v ) log ( \frac{log 6 }{log 2} - α v ) + ( α v - \frac{log 3}{log 2} ) log ( α v - \frac{log 3}{log 2} ) ] \qquad ( 17 )$$

where ν is a critical exponent of a correlation length. Also, αₘₐₓ and αₘᵢₙ are given by the following equations (18) and (19), respectively:

$$\begin{matrix}{α}_{max} = \frac{log 6}{v log 2} & ( 18 ) \\ {α}_{min} = \frac{log 3}{v log 2} & ( 19 )\end{matrix}$$

f(α) given by the equation (17) can be written as follows using αₘₐₓ and αₘᵢₙ:

$$\begin{matrix}f ( α ) = \frac{1}{v} - \frac{1}{log 2} \{ ( {α}_{max} - α ) log [ ( {α}_{max} - α ) v ] + \\ ( α - {α}_{min} ) log [ ( α - {α}_{min} ) v ] \} \\ = \frac{1}{v} - \frac{1}{log 2} [ ( {α}_{max} - α ) log ( {α}_{max} - α ) + \\ ( α - {α}_{min} ) log ( α - {α}_{min} ) + ( {α}_{max} - {α}_{min} ) log v ]\end{matrix} \qquad ( 20 )$$

This function takes a value 1/ν when α=αₘᵢₙ and α=αₘₐₓ. Since it is apparent that f(αₘᵢₙ)=f(αₘₐₓ)=0, f(α) of the blood flow velocity distribution is taken as a possible theoretical formula for comparison of the above equation in which the first term is taken as zero. That is, f(α) of the blood flow velocity distribution is given as follows:

$$f ( α ) = - \frac{1}{log 2} [ ( {α}_{max} - α ) log ( {α}_{max} - α ) + ( α - {α}_{min} ) log ( α - {α}_{min} ) + ( {α}_{max} - {α}_{min} ) log v ] \qquad ( 21 )$$

The following is derived from the above equations (18) and (19):

$${α}_{max} - {α}_{min} = \frac{1}{v} \qquad ( 22 )$$

By placing the equation (22) in the equation (21), f(α) will be expressed as follows:

$$f ( α ) = \frac{1}{log 2} [ ( {α}_{max} - {α}_{min} ) log ( {α}_{max} - {α}_{min} ) - ( {α}_{max} - α ) log ( {α}_{max} - α ) - ( α - {α}_{min} ) log ( α - {α}_{min} ) ] \qquad ( 23 )$$

The coefficient 1/log 2 in the equation (23) is peculiar to the hierarchical resistance network and does not provide any correct height of f(α) since the first term of the equation (17) is taken as zero. On this account, the coefficient 1/log 2 is taken as f₀ and the value of f₀ in the blood flow velocity distribution is selected from the conditions f(α) should satisfy. The maximum value f(α₀) of the function f(α) should be equal to the dimension of support of the distribution. Since the dimension is 2 in the blood flow velocity distribution, the following should holds:

f(α₀)=2 (24)

f(α) given by the equation (23) is symmetric with respect to its maximum value, the following holds:

$${α}_{0} = \frac{{α}_{max} + {α}_{min}}{2} \qquad ( 25 )$$

Therefore, the following is derived from the equation (24):

$${f}_{0} ( {α}_{max} - {α}_{min} ) log ( {α}_{max} - {α}_{min} ) - ( {α}_{max} - {α}_{0} ) log ( {α}_{max} - {α}_{0} ) - ( {α}_{0} - {α}_{min} ) log ( {α}_{0} - {α}_{min} ) ] = 2 \qquad ( 26 )$$

Calculation of the equation (26) results in the following:

f₀(αₘₐₓ−αₘᵢₙ)log 2=2 (27)

### Therefore,

$${f}_{0} = \frac{2}{( {α}_{max} - {α}_{min} ) log 2} \qquad ( 28 )$$

In this analysis, f₀ is taken as 1/log b where b is as follows:

b=2^{(α}^{ₘₐₓ}^{-α}^{ₘᵢₙ}^{)/2} (29)

Finally, the multifractal spectrum theoretically evaluated is given by the following equation (30):

$$f ( α ) = \frac{1}{{2}^{( {α}_{max} - {α}_{min} ) / 2}} [ ( {α}_{max} - {α}_{min} ) log ( {α}_{max} - {α}_{min} ) - ( {α}_{max} - α ) log ( {α}_{max} - α ) - ( α - {α}_{min} ) log ( α - {α}_{min} ) ] \qquad ( 30 )$$

As will be known from the above discussion, a theoretical formula for f(α) to be compared can be determined based on αₘₐₓ and αₘᵢₙ. For calculating the evaluation index 3, the domain of the variable α is resealed from [αₘᵢₙ, αₘₐₓ] to [0, 1]. That is, the variable is changed to α′ using the following:

$$α ↦ {α}^{′} = \frac{α - {α}_{min}}{{α}_{max} - {α}_{min}} \qquad ( 31 )$$

With integration of the square of a difference between the theoretical formula with the new variable

\tilde{f}(α′)

and actual f(α′), that is,

$$I = {\int }_{0}^{1} {[ f ( {α}^{′} ) - \tilde{f} ( {α}^{′} ) ]}^{2} d {α}^{′} \qquad ( 32 )$$

the deviation from the theoretical formula can be evaluated without dependence upon the domain of α. Further, for the evaluation index 3 to be 1 when the deviation from the theoretical formula is maximum, the integrated value was rescaled with a product Iₘₐₓ resulting from a completely asymmetric spectrum of f(α′)=2α′ (at this time, α₀=αₘᵢₙ or α₀=αₘₐₓ). In fact, Iₘₐₓ can be calculated based on the equation (30) as follows:

$${I}_{max} = \frac{2}{9 {( log 2 )}^{2}} [ 15 - 9 log 2 + 6 {( log 2 )}^{2} - {π}^{2} ] \qquad ( 33 )$$

Finally, the evaluation index 3 is defined as follows:

$$Evaluation index 3 = \frac{9 {( log 2 )}^{2} {\int }_{0}^{1} {[ f ( {α}^{′} ) - \tilde{f} ( {α}^{′} ) ]}^{2} d ( {α}^{′} )}{2 [ 15 - 9 log 2 + 6 {( log 2 )}^{2} - {π}^{2} ]} \qquad ( 34 )$$

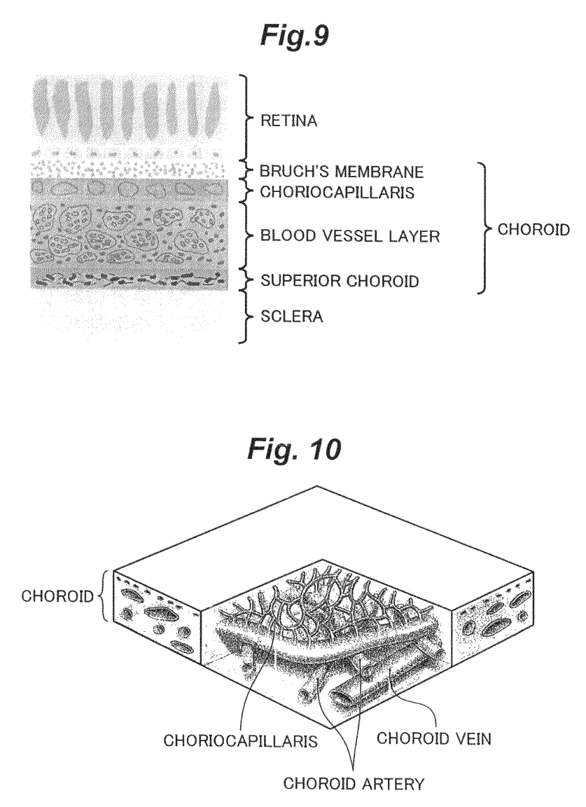

The aforementioned ocular fundus blood flowgraphy system was used to examine the choroid vascular network in a macular area of the eyeball of an examinee as will be described below. FIG. 8 is a horizontal sectional view of the eyeball, and FIG. 9 is a fragmentary sectional view of the eye, showing the retina, choroid and sciera. FIG. 10 shows an example of the choroid vascular network (a partially modified version of the illustration on page 26 of “The Atlas of Human Diseases—New Edition” under the editorship of Kazuyoshi Yamaguchi, Kodansha, Nov. 20, 2000).

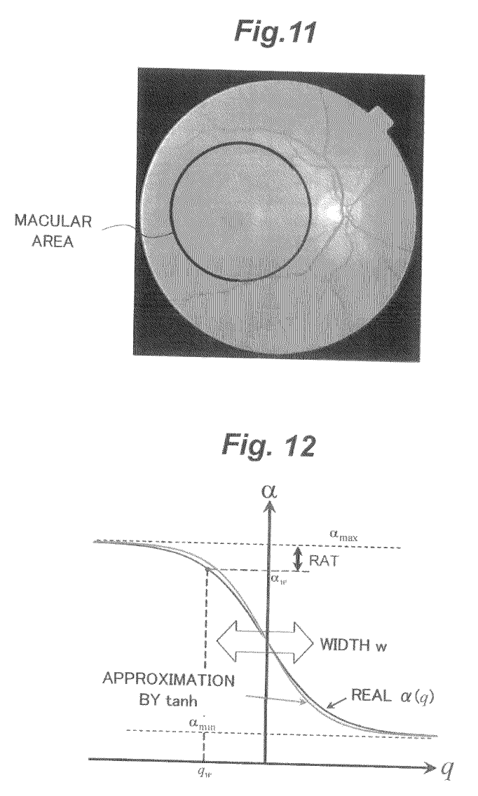

First, the ocular fundus is imaged using the fundus camera. FIG. 11 shows an ocular fundus image captured by the fundus camera, by way of example. A macular area is indicated within a circle. In the ocular fundus image, the thick blood vessels appearing mainly outside the circle are of the retina. No retinal vessels are found in the circle-enclosed area. The fundus camera is positioned for one of the focuses of its imaging lens to coincide with the light-incident surface of the two-dimensional CCD sensor as the photodetector 3. The laser light 6 having a wavelength in the near-infrared region is generated by the laser light source 1 and irradiated to the ocular fundus through the imaging lens 2. The laser light 6 incident upon the ocular fundus travels divergently into the ocular fundus and arrives at the choroid vascular network. At this time, the scattered light rays 8 by the choroid vascular network and coming out to the front of the eyeball (observation side) is passed through the imaging lens 2 again for focusing on the light-incident surface of the two-dimensional CCD sensor. An analog signal output from the two-dimensional CCD camera is converted into a digital signal by digital conversion. Calculation is performed by the personal computer system using this digital signal to make real-time measurement of the blood flow velocity distribution in the choroid vascular network in the macular area. This measurement is effected for several heart beats.

The real-time blood flow velocity distribution data measured for several heart beats are used to calculate a mean blood flow velocity distribution for one heart beat to provide a composite map. The macular area to be analyzed is extracted from all these composite map data. At this time, an area size from which a larger number of divisors (types of divisional boxes) is selected for an improved accuracy of the multifractal analysis. More specifically, the area size should be 240×240 or 180×180, for example.

For the result of analysis not to depend upon a variation of conditions during measurement, linear transformation is made of the blood flow velocity data so that the maximum and minimum values of the blood flow velocity are 4 and 1, respectively. The blood flow velocity data thus rescaled is used to calculate α₀, αₘᵢₙ, and αₘₐₓ, and evaluation order q_{w} and evaluation function width w for an improved efficiency of the calculation. By using the evaluation function, the measured and theoretical values of f(α) are calculated efficiently. The result of calculation is displayed on the display 5.

The evaluation order q_{w} and evaluation function width w will be explained below with reference to FIG. 12. The analysis is so adapted that the multifractal spectrum f(α) can give data to α as evenly as possible as will be described below. First, it is assumed herein that the relation between the values q and α is roughly as follows (see FIG. 12):

$$α = \frac{{α}_{max} + {α}_{min}}{2} - \frac{{α}_{max} - {α}_{min}}{2} tanh ( q / w ) \qquad ( 35 )$$

In order to determine a width w, q_{w} is determined. q_{w} provides a point α_{w} at a distance of RAT times of (αₘₐₓ−αₘᵢₙ) from αₘₐₓ. Since this calculation is to provide points a nearly uniformly, the relation between q and α may not be determined so exactly. A width w of the tan h function is determined using the following equation (36) resulted from solution, with the values q_{w} and α_{w}, of the equation (35):

$$w = \frac{2 {q}_{w}}{log ( \frac{{α}_{max} - {α}_{w}}{{α}_{w} - {α}_{min}} )} \qquad ( 36 )$$

Therefore, a value q for determining an even a is calculated using the following equation (37) derived from the equation (35) and f(α) is determined for q.

$$q = \frac{w}{2} log ( \frac{{α}_{max} - α}{α - {α}_{min}} ) \qquad ( 37 )$$

The results of the examinations actually effected on the examinees will be explained below.

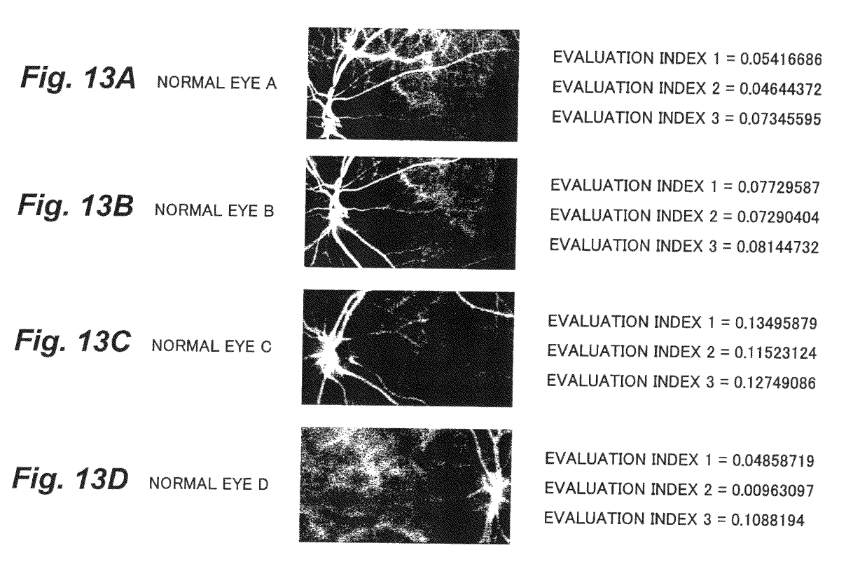

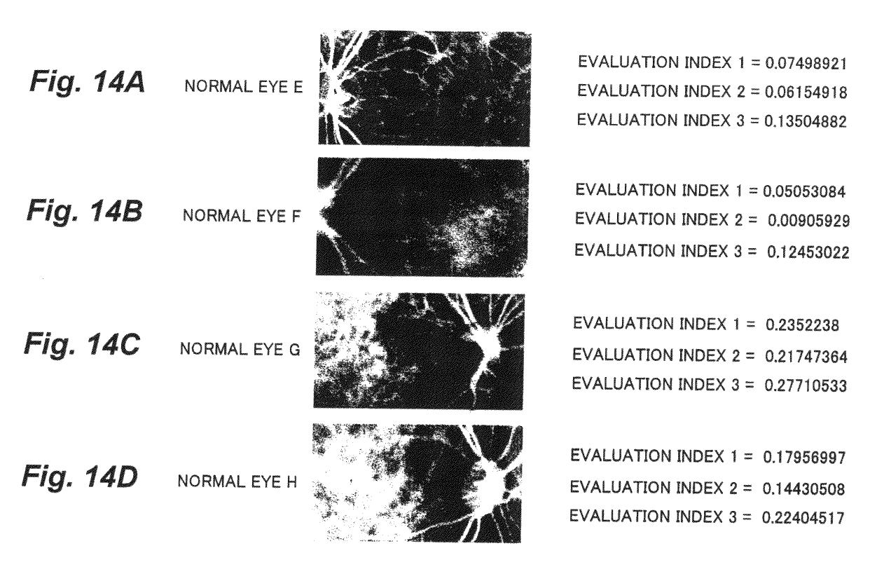

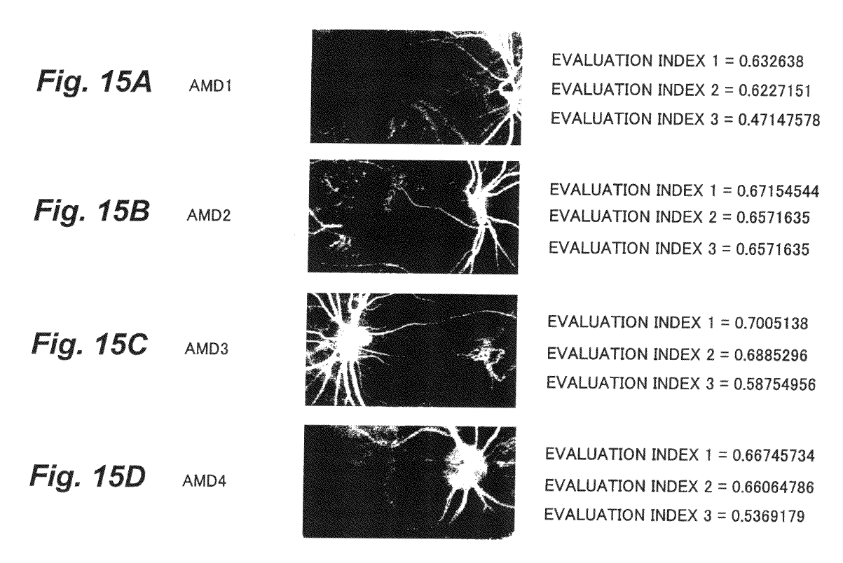

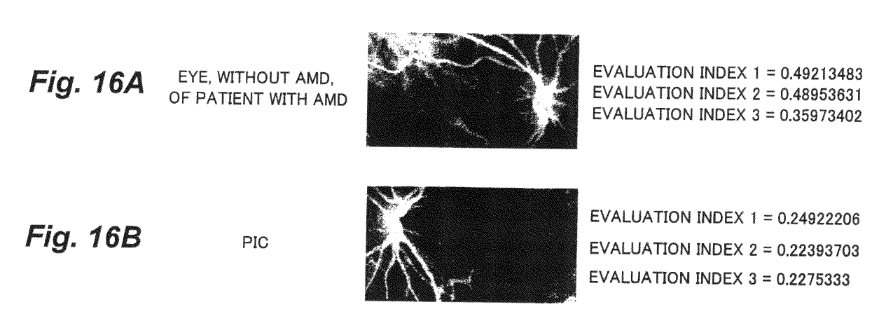

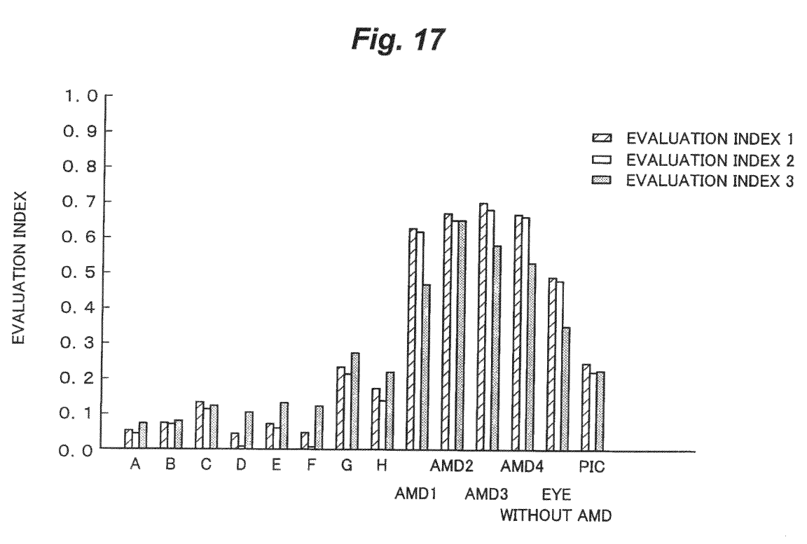

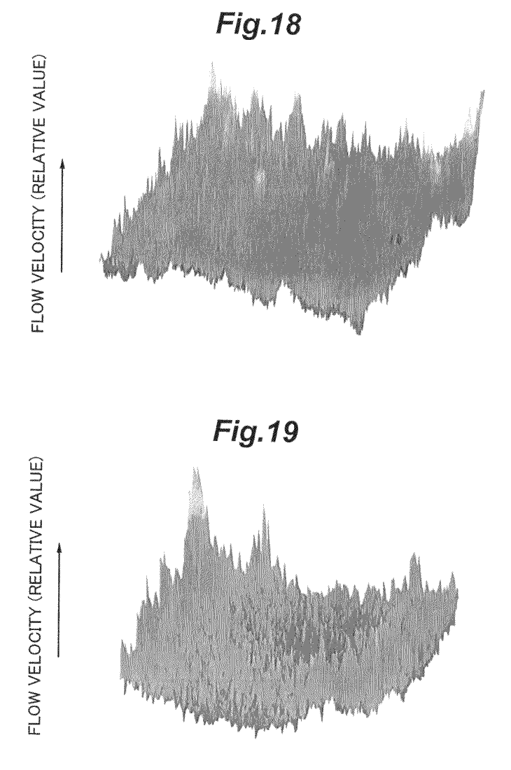

The examinations were made of examinees including eight examinees having normal eyes (will be referred to with alphabets A to H, respectively), five examinees with AMD (age-related macular degeneration) disease and one examinee with PIC (punctate inner choroidopathy) disease. The choroid vascular network in the macular area was examined by the aforementioned method to determine the evaluation indexes 1 to 3. It should be noted here that four (AMD1 to AMD4) of the five examinees with AMD disease had AMD at both eyes and the remaining one examinee with AMD disease had AMD at one eye. The four examinees with AMD at both eyes were examined at one of their eyes, and one examinee with AMD at one eye was examined at the other eye with no AMD. FIGS. 13A to 13D, FIGS. 14A to 14D, FIGS. 15A to 15D and FIGS. 16A and 16B show ocular fundus images, captured by the laser speckle flowgraphy, of these fourteen examinees, each with evaluation indexes 1 to 3. FIG. 17 graphically shows values of the evaluation indexes 1 to 3 of the fourteen examinees. As will be seen from the results of examination, all the evaluation indexes 1 to 3 show the same tendency but the evaluation index 3 responds to the extent of multifractal property most acutely. FIG. 18 shows the multifractal spectrum of the examinee E with normal eyes, and FIG. 19 shows the multifractal spectrum of the examinee AMD1 with AMD disease. In FIGS. 18 and 19, the vertical axis shows the flow velocity (relative value).

It will be known from FIGS. 13A to 13D, FIGS. 14A to 14D, FIGS. 15A to 15D and FIGS. 16A and 16B that all the evaluation indexes 1 to 3 of the examinees AMD1 to AMD4 with AMD disease are apparently larger than the evaluation indexes 1 to 3 of the examinees A to H with normal eyes and the blood flow distribution in the choroid vascular network in the macular area of the examinees AMD1 to AMD 4 deviates largely from the multifractal distribution. Conversely, the above result of examination reveals that the presence or absence, and extent in seriousness, of an abnormal blood flow, in the choroid vascular network in the macular area of the examinees can simply be examined based on the evaluation indexes 1 to 3. For example, in case the evaluation index 3 is 0.3 or less, the blood flow in the choroid vascular network in the macular area may be determined to be normal. In case the evaluation index 3 is 0.5 or more, the blood flow in the choroid vascular network in the macular area may be determined to be abnormal. In the latter case, it is possible to diagnose the examinee as having an eye disease or a disease in which such abnormal blood flow occurs by appropriately effecting the physiological function tests, slit-lamp microscopy, funduscopy, perimetry, fluorescein fundus angiography, electrophysiological study, etc.

Also, the evaluation index 3 of the normal eye, without AMD, of the examinee 5 with AMD disease at one eye is about 0.36 and this value is intermediate between the value of the evaluation index 3 of the examinees A to H with normal eyes and that of the evaluation index 3 of the examinees AMD1 to AMD4 all with AMD disease at both eyes, which suggests that the normal eye of the examinee 5 will possibly suffer from AMD.

As above, this embodiment permits to measure a blood flow velocity distribution in the vascular network in a region of interest of an examinee and make multifractal analysis of the blood flow velocity distribution to determine a pre-selected evaluation index, to thereby examine simply and accurately the blood flow in the vascular network in a noncontact, noninvasive manner and accurately measure the presence or absence, and extent in seriousness, of an abnormal blood flow. By adopting other appropriate studies for the examinee thus found to have the abnormal blood flow, the disease can be diagnosed more easily and accurately in a short time than with the conventional examination methods. Also, the result of diagnosis varies less from one doctor to another than before.

In the foregoing, the present invention has been described in detail concerning one preferred embodiment thereof and example of the embodiment. However, the present invention is not limited to the embodiment and example but can be modified in various manners based on the technical idea of the present invention.

For example, the numerical values, constructions, evaluation indexes, etc. in the foregoing description of the embodiment and example are given just as examples. Different numerical values, constructions, evaluation indexes, etc. from the above may be used as necessary.

As having been described in the foregoing, the present invention permits to make noncontact, noninvasive measurement of the blood flow velocity distribution in a vascular network with the use of the laser speckle flowgraphy or the like. Also, according to the present invention, the multifractal analysis of the blood flow velocity distribution in a vascular network can automatically be effected simply in a short time with the use of an arithmetic unit. By making quantitative evaluation of a deviation from the multifractal distribution through the multifractal analysis, the blood flow in the vascular network can be examined simply and accurately to find simply and accurately the presence or absence, and extent in seriousness, of an abnormal blood flow. Based on the result of examination, other examination methods can appropriately be adopted in combination with the examination method according to the present invention to make easy and accurate diagnosis of a disease with an abnormal blood flow in a vascular network.

1. An examination system for examining blood flow in a vascular network, comprising:
means for determining a blood flow velocity distribution in the vascular network by multifractal analysis; and
means for detecting a deviation of the blood flow velocity distribution from a multifractal distribution.

2. (canceled)

3. The examination system according to claim 1, wherein the blood flow velocity distribution in the vascular network is determined by laser speckle flowgraphy.

4. The examination system according to claim 1, wherein the vascular network is a choroid vascular network.

5. An examination system for examining the blood flow in a vascular network, the system comprising:
a laser light source to irradiate laser light to the vascular network;
a photodetector to detect scattered light rays resulting from irradiation of the laser light to the vascular network; and
an arithmetic unit for determining a blood flow velocity distribution in the vascular network on the basis of an output signal from the photodetector and making multifractal analysis of the blood flow velocity distribution to detect a deviation of the blood flow velocity distribution from a multifractal distribution.

6. (canceled)

7. A method for examining the blood flow in a vascular network, the method comprising:
irradiating the vascular network with a laser light;
detecting scattered light rays resulting from irradiation of the vascular network with the laser light; and
determining a blood flow velocity distribution in the vascular network on the basis of the scattered light rays and analyzing the blood flow velocity distribution by multifractal analysis to detect a deviation of the blood flow velocity distribution from a multifractal distribution.

8. The examination system according to claim 1, wherein the blood flow velocity distribution in the vascular network is determined by a Doppler Global Velocimeter method.

9. The examination system according to claim 1, wherein the vascular network is within an animal having a closed circulatory system.

10. The examination system according to claim 1, wherein the vascular network is in a mammal.

11. The examination system according to claim 1, wherein the vascular network is in a human being and is selected from the group consisting of a choroid vascular network, a retinal vascular network, a vascular network in an upper bodily portion, a pulmonary vascular network, a hepatic vascular network, a gastric vascular network, a splenic vascular network, an intestinal vascular network, a kidney vascular network, and a vascular network in a lower bodily portion.

12. The method of claim 7, wherein the laser light has a wavelength band ranging from near-infrared light to visible light.

13. The method of claim 7, wherein the detecting of the scattered light rays is accomplished using a two-dimensional image sensor selected from the group consisting of a CCD sensor, a MOS sensor and an image pickup tube.

14. The examination system according to claim 7, wherein the blood flow velocity distribution in the vascular network is determined by the laser speckle flowgraphy.

15. The examination system according to claim 7, wherein the vascular network is a choroid vascular network.
